(19) United States
(12) Patent Application Publication
(10) Pub. No.: US 2026/0207034 A1
(43) Pub. Date: Jul. 23, 2026

(54) CONTROL DEVICE, ENDOSCOPE SYSTEM, AND POSITION DETECTION METHOD

(71) Applicant: OLYMPUS MEDICAL SYSTEMS CORP., Tokyo (JP)

(72) Inventor: Sayuri YAMAMOTO, Tokyo (JP)

(73) Assignee: OLYMPUS MEDICAL SYSTEMS CORP., Tokyo (JP)

(21) Appl. No.: 19/571,654

(22) Filed: Mar. 19, 2026

Related U.S. Application Data
(63) Continuation of application No. PCT/JP2023/033984, filed on Sep. 19, 2023.

(51) Int. Cl. A61B 1/00; A61B 34/20

(52) U.S. Cl. CPC A61B 1/00158; A61B 1/00071; A61B 34/20; A61B 2034/2051

(57) ABSTRACT

A control device includes a connection interface and processing circuitry. The processing circuitry selects candidate coordinates of a target element, which is one a magnetic field generation element or a magnetic field detection element. It calculates a candidate vector based on coordinates of a reference element (the other of the magnetic field generation element or the magnetic field detection element), the candidate coordinates, and a detection signal received via the connection interface from the magnetic field detection element in response to detection of a magnetic field generated by the magnetic field generation element. It calculates a vector norm error based on the candidate vector, updates maximum likelihood coordinates to the candidate coordinates when the vector norm error is smaller than a minimum value of previously-calculated vector norm errors, and selects new candidate coordinates when determining that the vector norm error is not within a first range.

### CROSS REFERENCE TO RELATED APPLICATIONS

This application is a continuation of International Application No. PCT/JP2023/033984, filed on Sep. 19, 2023, the entire contents of which are incorporated herein by reference.

### BACKGROUND

### Technical Field

The present disclosure relates to a control device, an endoscope system, and a position detection method for detecting a position of an endoscope using a magnetic field.

### Related Art

Conventionally, endoscope devices are used in various fields such as medical fields, industrial fields, and academic fields. When an endoscope is in use, an insertion portion is inserted into a subject to observe an inside of the subject.

For example, in the medical field, there is a growing demand for examination using colonoscopes. The difficulty of an insertion procedure in colonoscopy is high. Therefore, a technology has been proposed that uses an endoscope insertion shape detection device (UPD) to detect a shape of an insertion portion of an endoscope, thereby enabling smooth insertion procedures.

In the endoscope insertion shape detection device, for example, a plurality of transmission coils are arranged in the insertion portion of the endoscope, and a plurality of reception coils are disposed in an antenna outside a body cavity. Then, a magnetic field is generated by the transmission coils, and a voltage generated in the reception coils by the generated magnetic field is detected to estimate positions and directions of the transmission coils. If the positions and the directions of the plurality of transmission coils arranged in the insertion portion can be estimated respectively, the shape of the insertion portion of the endoscope can be estimated.

For example, Japanese Patent No. 5231681 describes an example of a shape detection device using a magnetic field. The shape detection device calculates candidate vectors indicating directions of transmission coils based on a set of search coordinates (assumed three-dimensional coordinates) of the transmission coils and a measurement voltage of a reception coil group. The shape detection device then determines as a solution (estimated coordinates) a coordinate set that minimizes a sum of errors between estimated electromotive voltages of other reception coil groups obtained based on the search coordinate set and the candidate vectors and measurement voltages measured in the other reception coil groups. Using this method can reduce a calculation amount and a search amount, thereby rapidly obtaining an estimation result (the positions and the directions of the transmission coils).

### SUMMARY

According to aspects of the present disclosure, a control device is provided, which includes a connection interface and processing circuitry. The processing circuitry is configured to select candidate coordinates of a target element. The target element is one of a magnetic field generation element or a magnetic field detection element. The processing circuitry is further configured to calculate a candidate vector based on coordinates of a reference element, the candidate coordinates, and a detection signal received via the connection interface from the magnetic field detection element in response to detection of a magnetic field generated by the magnetic field generation element. The reference element is the other of the magnetic field generation element or the magnetic field detection element. The processing circuitry is further configured to calculate a vector norm error based on the candidate vector. The processing circuitry is further configured to update maximum likelihood coordinates to the candidate coordinates when the vector norm error is smaller than a minimum value of one or more previously-calculated vector norm errors. The processing circuitry is further configured to determine whether the vector norm error is within a first range. The processing circuitry is further configured to select new candidate coordinates when the vector norm error is not within the first range.

According to aspects of the present disclosure, further provided is an endoscope system that includes an endoscope, a magnetic field generation element, a magnetic field detection element, and a control device. The magnetic field generation element is configured to generate a magnetic field. The magnetic field detection element is configured to detect the magnetic field and output a detection signal. The control device includes a connection interface and processing circuitry. The processing circuitry is configured to select candidate coordinates of a target element. The target element is one of the magnetic field generation element or the magnetic field detection element. The processing circuitry is further configured to calculate a candidate vector based on coordinates of a reference element, the candidate coordinates, and the detection signal received via the connection interface from the magnetic field detection element in response to detection of the magnetic field generated by the magnetic field generation element. The reference element is the other of the magnetic field generation element or the magnetic field detection element. The processing circuitry is further configured to calculate a vector norm error based on the candidate vector. The processing circuitry is further configured to update maximum likelihood coordinates to the candidate coordinates when the vector norm error is smaller than a minimum value of one or more previously-calculated vector norm errors. The processing circuitry is further configured to determine whether the vector norm error is within a first range. The processing circuitry is further configured to select new candidate coordinates when the vector norm error is not within the first range.

According to aspects of the present disclosure, further provided is a position detection method that includes generating a magnetic field by a magnetic field generation element. The position detection method further includes outputting a detection signal from a magnetic field detection element in response to detection of the magnetic field. The position detection method further includes selecting candidate coordinates of a target element. The target element is one of the magnetic field generation element or the magnetic field detection element. The position detection method further includes calculating a candidate vector based on coordinates of a reference element, the candidate coordinates, and the detection signal. The reference element is the other of the magnetic field generation element or the magnetic field detection element. The position detection method further includes calculating a vector norm error based on the candidate vector. The position detection method further includes updating maximum likelihood coordinates to the candidate coordinates when the vector norm error is smaller than a minimum value of one or more previously-calculated vector norm errors. The position detection method further includes determining whether the vector norm error is within a first range. The position detection method further includes selecting new candidate coordinates when the vector norm error is not within the first range.

### BRIEF DESCRIPTION OF DRAWINGS

FIG. 1 shows a configuration of an endoscope system of a first embodiment of the present disclosure.

FIG. 2 shows a configuration example in which a magnetic field generation element is disposed in an endoscope and a magnetic field detection element is disposed in an antenna outside the endoscope in the first embodiment.

FIG. 3 shows a configuration example of the magnetic field detection element in the first embodiment.

FIG. 4 shows a configuration example in which a signal detected by a reception coil of the magnetic field detection element is amplified and converted into a digital signal and is transmitted to a magnetic field detection unit in the first embodiment.

FIG. 5 shows a configuration of a position detection system in the first embodiment.

FIG. 6 shows an example in which one of a plurality of sets of lattice point coordinates in a predetermined spatial range centered on search center coordinates is selected as candidate coordinates in the first embodiment.

FIG. 7 is a flowchart showing a part of transmission coil coordinate estimation processing in the first embodiment.

FIG. 8 is a flowchart showing stage 1 processing, which is a part of the transmission coil coordinate estimation processing in the first embodiment.

FIG. 9 is a flowchart showing stage 2 processing, which is a part of the transmission coil coordinate estimation processing in the first embodiment.

FIG. 10 shows a configuration example in which a magnetic field detection element is disposed in an endoscope and a magnetic field generation element is disposed in an antenna outside the endoscope in a second embodiment of the present disclosure.

FIG. 11 shows a configuration example in which a signal detected by a reception coil of a magnetic field detection element is amplified so as to be able to change an amplification factor and converted into a digital signal, and is transmitted to a magnetic field detection unit in a third embodiment of the present disclosure.

FIG. 12 shows a configuration of a position detection system in a fourth embodiment of the present disclosure.

FIG. 13 is a flowchart showing stage 1 processing, which is a part of transmission coil coordinate estimation processing in the fourth embodiment of the present disclosure.

FIG. 14 is a flowchart showing stage 2 processing, which is a part of the transmission coil coordinate estimation processing in the fourth embodiment of the present disclosure.

### DESCRIPTION OF EMBODIMENTS

In the technology described in Japanese Patent No. 5231681, if the calculation result of the candidate vectors deviates significantly from a true value, the solution may fall into a local minimum (a local minimum value of an evaluation function that differs from the true value), and converge to a point completely different from the true value. Convergence of the solution to a point completely different from the true value may occur, for example, when the transmission coils are disposed at near points of the reception coils. Therefore, the technology described in Japanese Patent No. 5231681 is not suitable for a case where the antenna in which the reception coils are disposed is disposed at the near point of the endoscope.

On the other hand, in principle, the greater a distance between a transmission coil, which is a magnetic field generation element, and a reception coil, which is a magnetic field detection element, the greater an influence of noise and the lower a detection accuracy.

Therefore, there is a need for a technology that can improve a detection accuracy without a solution converging at a point completely different from a true value even when the distance between the magnetic field generation element and the magnetic field detection element is reduced.

Hereinafter, embodiments of the present disclosure will be described with reference to the drawings. However, the present disclosure is not limited by the embodiments described below.

Note that in the illustration of the drawings, the same or corresponding elements are attached with the same reference signs as appropriate. In addition, the respective drawings are schematically shown, and care should be taken to the fact that a relationship among the lengths of the respective elements, the ratio of the lengths among the respective elements, the number of the respective elements, and the like in one drawing are sometimes different from the actual ones for simplification of the description. Furthermore, the respective drawings sometimes include parts in which the relationships and ratios among the lengths of the elements, the number and the like are different.

### First Embodiment

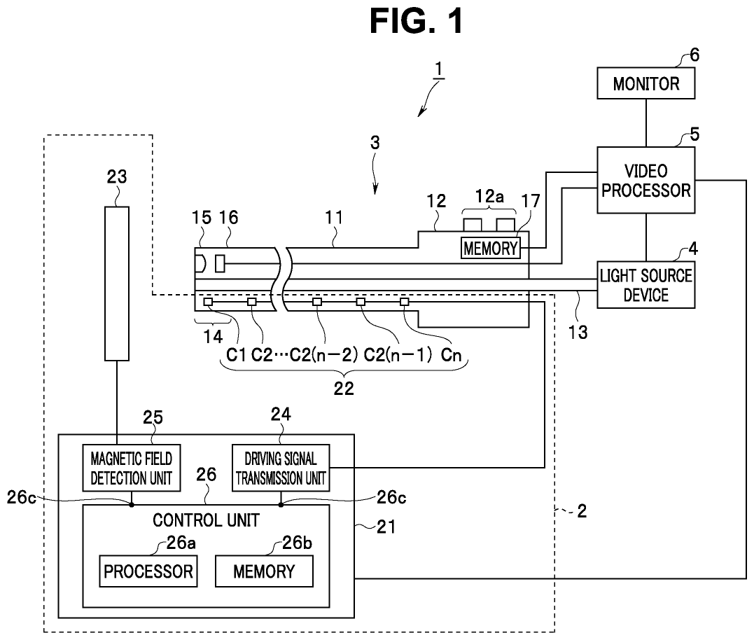

FIG. 1 to FIG. 9 show a first embodiment of the present disclosure. FIG. 1 shows a configuration of an endoscope system 1 of the first embodiment.

As shown in FIG. 1, the endoscope system 1 includes a position detection system 2, an endoscope 3, a light source device 4, a video processor 5, and a monitor 6.

The endoscope 3 includes an insertion portion 11 to be inserted into a subject, and an operation portion 12 provided at a proximal end of the insertion portion 11.

The insertion portion 11 is assumed to be of a type including a flexible tube portion and a bending portion, that is, a type whose shape changes. An objective lens 15 and an image pickup device 16 are disposed at a distal end portion 14 of the insertion portion 11.

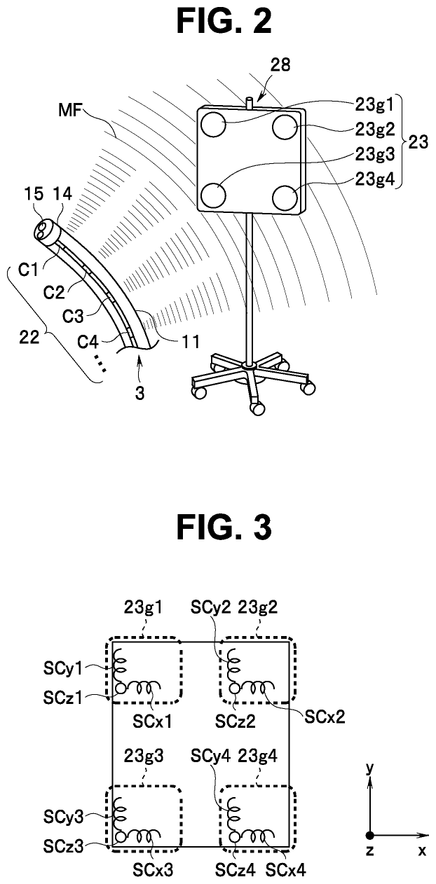

Furthermore, a magnetic field generation element 22 is disposed in the insertion portion 11 including the distal end portion 14. The magnetic field generation element 22 generates a magnetic field MF (see FIG. 2 and the like). The magnetic field generation element 22 includes, for example, a plurality of transmission coils C1 to Cn. The plurality of transmission coils C1 to Cn are arranged at a predetermined interval in order from a distal end side along a longitudinal direction of the insertion portion 11.

The operation portion 12 is provided with various switches 12a for operating the endoscope 3.

Furthermore, a memory 17 is provided, for example, in the operation portion 12 of the endoscope 3 (however, may be in a part other than the operation portion 12 as long as in the endoscope 3). The memory 17 stores various information related to the endoscope 3 in a non-volatile manner. Examples of information stored in the memory 17 include a model number and a serial number of the endoscope 3, parameters specific to the endoscope 3, and the like. The memory 17 is connected to the video processor 5, and transmits the various information related to the endoscope 3 to the video processor 5.

In addition, a bending operation knob for bending the bending portion, and the like are disposed on the operation portion 12.

A light guide 13 is disposed in the endoscope 3 from the distal end portion 14 of the insertion portion 11 along the longitudinal direction. The light source device 4 is connected to a proximal end of the light guide 13. The light guide 13 transmits illumination light supplied from the light source device 4 and irradiates the subject with the illumination light from the distal end portion 14.

An optical image of the subject irradiated with the illumination light is formed on the image pickup device 16 by the objective lens 15. The image pickup device 16 picks up the optical image of the subject and transmits an image pickup signal to the video processor 5.

The video processor 5 receives the image pickup signal from the image pickup device 16, and performs image processing on the image pickup signal based on the various information related to the endoscope 3 received from the memory 17, to generate a video signal. The video processor 5 transmits the video signal to the monitor 6.

The monitor 6 receives the video signal transmitted from the video processor 5 and displays an endoscopic image of the subject.

The position detection system 2 includes a position detection device 21, the magnetic field generation element 22, and a magnetic field detection element 23. In other words, the magnetic field generation element 22 is disposed in the endoscope 3, for example, and constitutes a part of the position detection system 2.

The position detection device 21 includes a driving signal transmission unit 24, a magnetic field detection unit 25, and a control unit 26. The control unit 26 includes connection interfaces 26c for transmitting signals to the driving signal transmission unit 24 and receiving signals from the magnetic field detection unit 25. The control unit 26 may be implemented as a separate control device configured to be operatively coupled to external devices such as the driving signal transmission unit 24 and the magnetic field detection unit 25 in a wired or wireless manner.

The driving signal transmission unit 24 transmits a driving signal for generating the magnetic field MF (an alternating signal for generating an alternating magnetic field, for example) to the magnetic field generation element 22, based on control of the control unit 26.

The driving signal transmission unit 24 simultaneously transmits alternating signals of frequencies different from one another to the transmission coils C1 to Cn, for example. As a result, the transmission coils C1 to Cn generate alternating magnetic fields of frequencies different from one another. In this case, the magnetic field MF generated by each of the transmission coils C1 to Cn can be distinguished based on the frequency of the alternating magnetic field.

In addition, the driving signal transmission unit 24 may sequentially transmit an alternating signal to one of the transmission coils C1 to Cn by switching the transmission coils C1 to Cn that transmit the alternating signal. In this case, the magnetic field MF generated by each of the transmission coils C1 to Cn can be distinguished based on a generation time of the alternating magnetic field.

FIG. 2 shows a configuration example in which the magnetic field generation element 22 is disposed in the endoscope 3, and the magnetic field detection element 23 is disposed in an antenna 28 outside the endoscope 3 in the first embodiment.

The magnetic field detection element 23 detects the magnetic field MF and transmits a detection signal. As shown in FIG. 2, the magnetic field detection element 23 is configured as the antenna 28 (a reception antenna in the example of FIG. 2) whose position can be moved by means of a caster, or the like, for example.

The antenna 28 is disposed in an examination room, for example, around an examination table (outside a body cavity) on which the subject is placed. Note that coordinates of the magnetic field detection element 23 after the antenna 28 is disposed are known.

The magnetic field detection element 23 includes a plurality of reception coil groups, and in the present embodiment, an example including four reception coil groups 23g1 to 23g4 is shown. The four reception coil groups 23g1 to 23g4 are disposed at different positions on the antenna 28.

FIG. 3 shows a configuration example of the magnetic field detection element 23 in the first embodiment. In FIG. 3, an xyz coordinate system is a three-dimensional orthogonal coordinate system. The xyz coordinate system is called a global coordinate system or a world coordinate system.

The reception coil group 23g1 includes a set of three coils: an x-direction reception coil SCx1 with a central axis parallel to an x-axis direction; a y-direction reception coil SCy1 with a central axis parallel to a y-axis direction; and a z-direction reception coil SCz1 with a central axis parallel to a z-axis direction. The x-direction reception coil SCx1, the y-direction reception coil SCy1, and the z-direction reception coil SCz1 detect magnetic field components in the mutually orthogonal the x-axis direction, the y-axis direction, and the z-axis direction, respectively.

In other words, the x-direction reception coil SCx1 generates an electromotive voltage corresponding to the magnetic field component in the x-axis direction of the magnetic field MF generated by the transmission coils C1 to Cn, at a position of the x-direction reception coil SCx1. The y-direction reception coil SCy1 generates an electromotive voltage corresponding to the magnetic field component in the y-axis direction of the magnetic field MF generated by the transmission coils C1 to Cn, at a position of the y-direction reception coil SCy1. The z-direction reception coil SCz1 generates an electromotive voltage corresponding to the magnetic field component in the z-axis direction of the magnetic field MF generated by the transmission coils C1 to Cn, at a position of the z-direction reception coil SCz1.

Similarly, the reception coil group 23g2 also includes a set of three coils: an x-direction reception coil SCx2; a y-direction reception coil SCy2; and a z-direction reception coil SCz2, and generates electromotive voltages corresponding to magnetic field components in respective directions.

Similarly, the reception coil group 23g3 also includes a set of three coils: an x-direction reception coil SCx3; a y-direction reception coil SCy3; and a z-direction reception coil SCz3, and generates electromotive voltages corresponding to magnetic field components in respective directions.

Similarly, the reception coil group 23g4 also includes a set of three coils: an x-direction reception coil SCx4; a y-direction reception coil SCy4; and a z-direction reception coil SCz4, and generates electromotive voltages corresponding to magnetic field components in respective directions.

Note that in the following, any of the reception coils SCx1 to SCz4 is simply referred to as a reception coil SC.

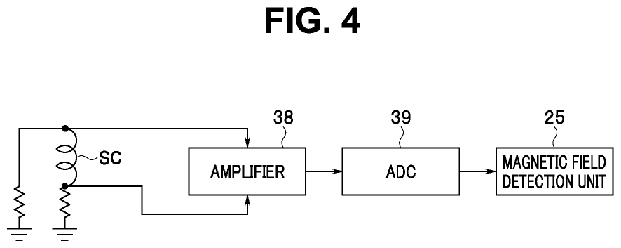

FIG. 4 shows a configuration example in which a signal detected by a reception coil SC of the magnetic field detection element 23 is amplified and converted into a digital signal and is transmitted to the magnetic field detection unit 25 in the first embodiment.

When the magnetic field MF passing through the reception coil SC changes over time, an electromotive voltage that changes over time is generated at both ends of the reception coil SC, and a current (alternating current) flows.

The electromotive voltage generated at the both ends of the reception coil SC is detected and amplified by an amplifier 38, converted into a digital signal by an analog-to-digital converter (ADC) 39, and transmitted to the magnetic field detection unit 25 as a detection signal.

At least one of the amplifier 38 or the ADC 39 may be provided in the position detection device 21 (in the magnetic field detection unit 25, for example), or may be provided in the antenna 28. For example, if the amplifier 38 and the ADC 39 are provided in the position detection device 21, the antenna 28 can be configured simply and inexpensively.

Furthermore, the electromotive voltage generated in the reception coil SC decreases as a distance between the transmission coils C1 to Cn and the reception coil SC increases. Therefore, if the amplifier 38 and the ADC 39 are provided in the antenna 28, for example, when a signal generated in the reception coil SC is transmitted from the antenna 28 to the position detection device 21, distortion of the signal and entering of noise in the signal can be reduced.

The magnetic field detection unit 25 separates by frequency (or by time), for example, an average received voltage amplitude for a certain time (obtained by Fourier transforming the voltage), i.e., the detection signal, which is transmitted from the reception coil SC through the amplifier 38 and the ADC 39, divides the detection signal into signals for each the reception coil groups 23g1, 23g2, 23g3, and 23g4, and transmits the signals to the control unit 26.

The control unit 26 is a control device, and may be implemented by processing circuitry including one or more processors. For instance, the control unit 26 includes processing circuitry including a processor 26a and a memory 26b. In other words, the control unit 26 is configured to perform functions of each unit described later, by the processor 26a such as an ASIC (application specific integrated circuit) and an FPGA (field programmable gate array), including a CPU (central processing unit) or the like, reading and executing a processing program stored in a storage device (or a storage medium) such as the memory 26b. However, the configuration of the control unit 26 is not limited to this, and for example, each unit may include dedicated electronic circuitry configured to perform respective functions.

The processor 26a processes the detection signals received from the magnetic field detection unit 25. The processor 26a performs processing to detect the positions and the directions of the transmission coils C1 to Cn based on the detection signals, as described later.

The arrangement of the transmission coils C1 to Cn in the insertion portion 11 along the longitudinal directions is known. Therefore, when the positions and the directions of the respective transmission coils C1 to Cn are obtained, the processor 26a can detect the shape of the insertion portion 11.

The position detection device 21 is connected to the video processor 5. The position detection device 21 generates, for example, an image indicating the shape of the insertion portion 11, insertion assistance information, or the like based on the detected shape of the insertion portion 11, and transmits the generated information to the video processor 5. The video processor 5 displays the information received from the position detection device 21 on the monitor 6 together with the endoscopic image to perform insertion assistance, and the like.

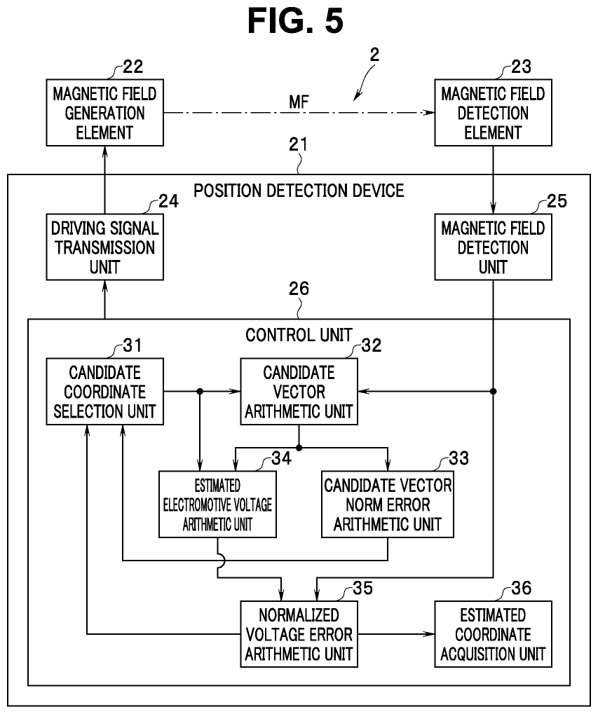

FIG. 5 shows a configuration of the position detection system 2 in the first embodiment.

As shown in FIG. 5, the control unit 26 includes, as function units, a candidate coordinate selection unit 31, a candidate vector arithmetic unit 32, a candidate vector norm error arithmetic unit 33, an estimated electromotive voltage arithmetic unit 34, a normalized voltage error arithmetic unit 35, and an estimated coordinate acquisition unit 36.

The candidate coordinate selection unit 31 selects candidate coordinates of the magnetic field generation element 22.

The candidate vector arithmetic unit 32 calculates a candidate vector Gₖ based on the coordinates of the magnetic field detection element 23, the candidate coordinates of the magnetic field generation element 22, and the detection signal received from the magnetic field detection unit 25.

The candidate vector norm error arithmetic unit 33 calculates a candidate vector norm error e₁ (for simplicity, also referred to as vector norm error) (first evaluation function) based on the candidate vector Gₖ.

The estimated electromotive voltage arithmetic unit 34 calculates an electromotive voltage (estimated electromotive voltage) estimated to be generated in the magnetic field detection element 23 based on the candidate vector Gₖ.

The normalized voltage error arithmetic unit 35 calculates an electromotive voltage error, which is an error between the estimated electromotive voltage and the measurement voltage obtained from the detection signal. Furthermore, the normalized voltage error arithmetic unit 35 normalizes the electromotive voltage error based on the measurement voltage, and calculates a normalized electromotive voltage error e₂ (second evaluation function).

The estimated coordinate acquisition unit 36 acquires current maximum likelihood coordinates as the estimated coordinates of the magnetic field generation element 22 when the normalized electromotive voltage error e₂ is within a second range.

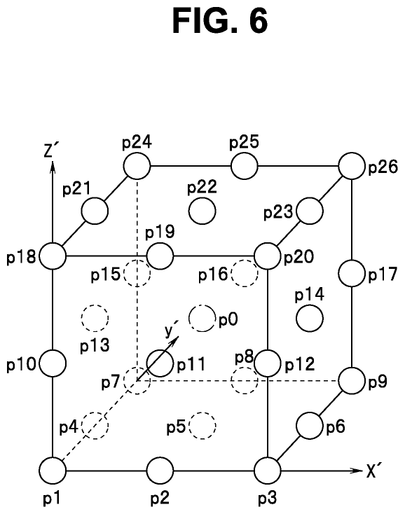

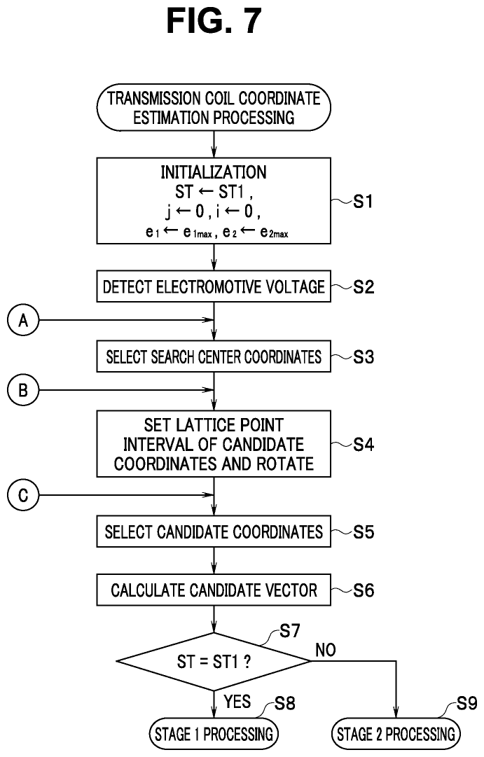

The more detailed operation of each function unit of the control unit 26 shown in FIG. 5 will be described with reference to FIG. 6 and according to flowcharts in FIG. 7 to FIG. 9.

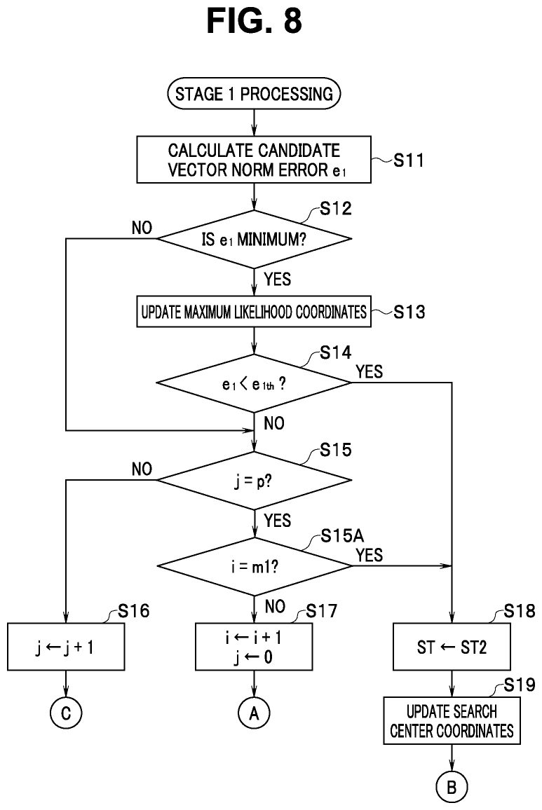

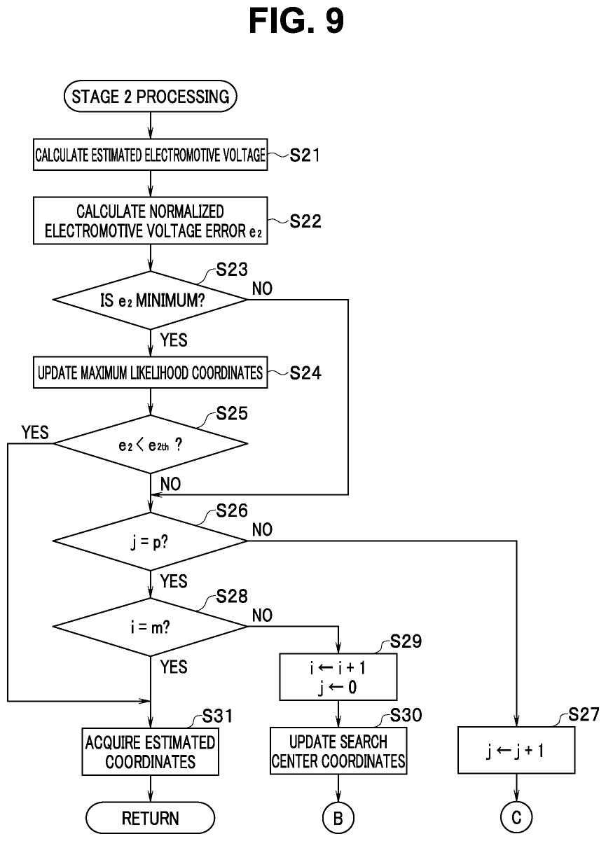

FIG. 6 shows an example in which one of a plurality of sets of lattice point coordinates p1 to p26 in a predetermined spatial range centered on search center coordinates p0 is selected as candidate coordinates in the first embodiment. FIG. 7 is a flowchart showing a part of transmission coil coordinate estimation processing in the first embodiment. FIG. 8 is a flowchart showing stage 1 processing, which is a part of the transmission coil coordinate estimation processing in the first embodiment. FIG. 9 is a flowchart showing stage 2 processing, which is a part of the transmission coil coordinate estimation processing in the first embodiment.

The transmission coil coordinate estimation processing shown in FIG. 7 to FIG. 9 is mainly performed by the processor 26a operating as each function unit shown in FIG. 5 in accordance with a computer program stored in the memory 26b.

In main processing not shown in the drawings, the processor 26a performs the processing shown in FIG. 7 each time the processor 26a receives the detection signal from the magnetic field detection unit 25.

When the processing shown in FIG. 7 is started, the processor 26a initializes each parameter (step S1).

In the transmission coil coordinate estimation processing shown in FIG. 7 to FIG. 9, the stage 1 processing in which the candidate vector Gₖ indicating the direction of the magnetic field generation element 22 (hereinafter, referred to as a transmission coil C, as appropriate) is estimated, and the stage 2 processing in which the position of the transmission coil C is estimated based on the candidate vector Gₖ estimated in stage 1 are mainly performed.

In the initialization in step S1, the processor 26a enters a value ST1, which indicates stage 1, into a parameter ST indicating a current stage. The processor 26a enters 0 into a counter j, which counts the number of processed lattice point coordinates (see FIG. 6) in the predetermined spatial range. The processor 26a enters 0 into a counter i, which counts the number of times the search center coordinates p0 are selected (that is, the number of times the lattice point coordinates (see FIG. 6) centered on the search center coordinates p0 are set), as described later. The processor 26a enters a maximum value e₁ₘₐₓ that can be taken as the candidate vector norm error e₁, into the candidate vector norm error e₁. The processor 26a enters a maximum value e₂ₘₐₓ that can be taken as the normalized electromotive voltage error e₂, into the normalized electromotive voltage error e₂.

Then, the processor 26a detects an electromotive voltage (measurement voltage) generated in the magnetic field detection element 23, from the detection signal transmitted from the magnetic field detection unit 25 (step S2).

The memory 26b stores in advance information on a range (entire range) of a space in which positions can be detected by the magnetic field detection element 23. Specifically, the memory 26b stores maximum and minimum values of each x, y, and z coordinates of the entire range, for example.

The processor 26a selects one of the three-dimensional coordinates of points included in the entire range stored in the memory 26b as the search center coordinates p0 (step S3).

The processor 26a sets a plurality of sets of lattice point coordinates in the predetermined spatial range centered on the search center coordinates p0. When setting the lattice point coordinates, the processor 26a sets a lattice point interval and rotates a lattice point coordinate system (step S4).

When performing the processing of step S4 for the first time after starting the processing of FIG. 7, the lattice point interval is set longer than when performing the processing of step S4 for the second time or later. Thereafter, when selecting new search center coordinates p0 (that is, when the maximum likelihood coordinates approach a true value (true coordinates of the magnetic field generation element 22) and it becomes acceptable to narrow a search range), the processor 26a reduces the predetermined spatial range and shortens the lattice point interval, and rotates the lattices by an appropriate angle (30° around a predetermined rotation axis, for example). However, it is not necessary to change the lattice point interval each time the processing of step S4 is performed. For example, the lattice point interval may be left unchanged while the current stage is stage 1, and may be changed after the current stage becomes stage 2 or later.

FIG. 6 shows an example of the lattice point coordinates set by the processor 26a. In the example shown in FIG. 6, in the x′y′z′ coordinate system, which is a local three-dimensional orthogonal coordinate system, 3×3×3 lattice point coordinates are set in an x′-axis direction, a y′-axis direction, and a z′-axis direction, respectively, with the search center coordinates p0 as the center.

Note that the lattice point coordinates to be set are not limited to 3×3×3, but may also be 5×5×5 or the like, for example. However, in order to obtain the estimated coordinates of the magnetic field generation element 22 rapidly with a small amount of calculation, it may be better not to increase the number of the lattice point coordinates.

A total of 27 lattice point coordinates, including the lattice point coordinates p1 to p26 and the search center coordinates p0, are disposed in the predetermined spatial range. Note that the x′y′z′ coordinate system, which is a local coordinate system, and the xyz coordinate system, which is a global coordinate system, are associated with each other, using a rotation matrix of the three-dimensional space.

The processor 26a (candidate coordinate selection unit 31) selects one of the lattice point coordinates as shown in FIG. 6, for example, as the candidate coordinates (step S5). When performing the processing of step S5 for the first time after starting the processing of FIG. 7, the search center coordinates p0 may be selected as the candidate coordinates, or one of the lattice point coordinates p1 to p26 may be selected as the candidate coordinates. In addition, when performing the processing of step S5 for the second time or later, since the search center coordinates p0 has already been selected as the candidate coordinates, one of the lattice point coordinates p1 to p26 is selected as the candidate coordinates.

The processor 26a (candidate vector arithmetic unit 32) calculates the candidate vector Gₖ based on the selected candidate coordinates (step S6).

Although the above description shows an example in which there are four sets of the reception coils, which are the reception coil groups 23g1 to 23g4, the following describes a more general case in which there are 1 sets of the reception coils (1 is an integer of 2 or more). In addition, any set number from 1 to 1 is represented by k (k=1, 2, . . . , 1). In the example shown in FIG. 2 and FIG. 3, 1=4.

An electromotive voltage vector vₖ created from the electromotive voltage generated in the magnetic field detection element 23 of a set k by the magnetic field MF generated from the transmission coil C is, in principle, expressed by (Equation 1) using a direction vector g (unit vector) of the transmission coil C, a matrix Bₖ, and a constant ζ.

$$\vec{{v}_{k}} = ξ {B}_{k} \vec{g} \qquad ( Equation 1 )$$

Here, the electromotive voltage vector vₖ is expressed by (Equation 2) using an electromotive voltage vₓₖ generated in the x-direction reception coil SCxk of the set k, an electromotive voltage v_{yk} generated in the y-direction reception coil SCyk of the set k, and an electromotive voltage v_{zk} generated in the z-direction reception coil SCzk of the set k.

$$\vec{{v}_{k}} = [ \begin{matrix}{v}_{xk} \\ {v}_{yk} \\ {v}_{zk}\end{matrix} ] \qquad ( Equation 2 )$$

The direction vector g of the transmission coil C is expressed by (Equation 3) using an x-direction component gₓ, a y-direction component g_{y}, and a z-direction component g_{z}.

$$\vec{g} = [ \begin{matrix}{g}_{x} \\ {g}_{y} \\ {g}_{z}\end{matrix} ] \qquad ( Equation 3 )$$

In addition, a value of the constant ζ is determined by a gain of an entire transmission and reception system including a transmission system (the magnetic field generation element 22, the driving signal transmission unit 24, and the like) that transmits the magnetic field MF, and a reception system (the magnetic field detection element 23, the magnetic field detection unit 25, and the like) that receives the magnetic field MF transmitted from the transmission system. The value of the constant ζ is determined by, for example, characteristics of the transmission coil C, characteristics of the reception coil, and the like. The constant ζ is stored in the memory 26b in advance.

The matrix Bₖ is expressed by (Equation 4) using a matrix Aₖ and a matrix Rₖ.

$${B}_{k} = {R}_{k}^{- 1} {A}_{k} \qquad ( Equation 4 )$$

The matrix Rₖ in (Equation 4) is expressed by (Equation 5).

$${R}_{k} = [ \begin{matrix}{r}_{xk}^{5} & 0 & 0 \\ 0 & {r}_{yk}^{5} & 0 \\ 0 & 0 & {r}_{zk}^{5}\end{matrix} ] \qquad ( Equation 5 )$$

The matrix components rₓₖ, r_{yk}, and r_{zk} in (Equation 5) are expressed by (Equation 6).

$${r}_{xk} = \vert {x}_{Rk} - {x}_{T} \vert \qquad ( Equation 6 )$$

$${r}_{yk} = \vert {y}_{Rk} - {y}_{T} \vert$$

$${r}_{zk} = \vert {z}_{R k} - {z}_{T} \vert$$

In (Equation 6), x_{Rk}, y_{Rk}, and z_{Rk} represent the x, y, and z coordinates of the reception coil SCk of the set k, respectively, and x_{T}, y_{T}, and z_{T} represent the x, y, and z coordinates of the transmission coil C, respectively. Therefore, rₓₖ, r_{yk}, and r_{zk} represent an x-direction distance, a y-direction distance, and a z-direction distance between the reception coil SCk and the transmission coil C, respectively.

In addition, the matrix Aₖ in (Equation 4) is expressed by (Equation 7).

$${A}_{k} = [ \begin{matrix}{A}_{11} & {A}_{21} & {A}_{31} \\ {A}_{12} & {A}_{22} & {A}_{32} \\ {A}_{13} & {A}_{23} & {A}_{33}\end{matrix} ] \qquad ( Equation 7 )$$

The matrix components in (Equation 7) are expressed by (Equation 8).

$${A}_{11} = 2 {( {x}_{Rk} - {x}_{T} )}^{2} - {( {y}_{R k} - {y}_{T} )}^{2} - {( {z}_{Rk} - {z}_{T} )}^{2} \qquad ( Equation 8 )$$

$${A}_{12} = 3 ( {y}_{Rk} - {y}_{T} ) ( {x}_{Rk} - {x}_{T} )$$

$${A}_{13} = 3 ( {z}_{Rk} - {z}_{T} ) ( {x}_{Rk} - {x}_{T} )$$

$${A}_{21} = 3 ( {x}_{Rk} - {x}_{T} ) ( {y}_{Rk} - {y}_{T} )$$

$${A}_{22} = 2 {( {y}_{Rk} - {y}_{T} )}^{2} - {( {x}_{R k} - {x}_{T} )}^{2} - {( {z}_{Rk} - {z}_{T} )}^{2}$$

$${A}_{23} = 3 ( {z}_{Rk} - {z}_{T} ) ( {y}_{Rk} - {y}_{T} )$$

$${A}_{31} = 3 ( {x}_{Rk} - {x}_{T} ) ( {z}_{Rk} - {z}_{T} )$$

$${A}_{33} = 2 {( {z}_{Rk} - {z}_{T} )}^{2} - {( {y}_{R k} - {y}_{T} )}^{2} - {( {x}_{Rk} - {x}_{T} )}^{2}$$

In other words, the matrix Aₖ is determined based on the xyz coordinates of the reception coil SCk and the xyz coordinates of the transmission coil C.

Therefore, the matrix Bₖ calculated using the matrices Aₖ and Rₖ according to (Equation 4) is determined based on the xyz coordinates of the reception coil SCk of the set k (the coordinates of the magnetic field detection element 23), and the candidate coordinates (the candidate coordinates of the magnetic field generation element 22) (each xyz coordinates of the transmission coil C).

In the principle described above, the candidate vector Gₖ expressed by (Equation 9) is obtained by calculating the matrix Bₖ using the candidate coordinates as the coordinates of the transmission coil C, and using the measurement value as the electromotive vector vₖ. The candidate vector Gₖ is obtained by multiplying the direction vector g of the candidate vector Gₖ (indicating the candidate of the direction of the transmission coil C) by the constant ζ.

$$\vec{{G}_{k}} = ξ \vec{g} = {B}_{k}^{- 1} \vec{{v}_{k}} \qquad ( Equation 9 )$$

Next, the processor 26a determines whether the value of the parameter ST is ST1, that is, whether the current stage is stage 1 (step S7).

Here, if the value of the parameter ST is ST1, the process proceeds to the stage 1 processing of FIG. 8 (step S8).

When entering the stage 1 processing of FIG. 8, the processor 26a (the candidate vector norm error arithmetic unit 33) calculates the candidate vector norm error e₁ expressed by (Equation 10) based on the candidate vector Gₖ (step S11). Note that in (Equation 10) (and (Equation 11) and (Equation 12) described later), the two vertical lines surrounding the vector represent the norm (specifically, a length of the vector (Euclidean norm)).

$${e}_{1} = {\sum }_{k = 1}^{l} {( \frac{‖ \vec{{G}_{k}} ‖}{ξ} - 1 )}^{2} \qquad ( Equation 10 )$$

Each time a new candidate vector norm error e₁ is calculated, the processor 26a compares the new candidate vector norm error e₁ with a minimum value of the candidate vector norm error e₁ calculated in the past (step S12).

In other words, the processor 26a stores the minimum value of the candidate vector norm error e₁ calculated after starting the processing shown in FIG. 7, for example, in the memory 26b. Then, the processor 26a compares the minimum value of the candidate vector norm error e₁ stored in the memory 26b with the newly calculated candidate vector norm error e₁.

Here, if the newly calculated candidate vector norm error e₁ is smaller than the minimum value of the candidate vector norm error e₁ calculated in the past, the processor 26a updates the maximum likelihood coordinates (the coordinates estimated to be closest to the true value (true coordinates of the magnetic field generation element 22) at the present time point) to the current candidate coordinates (step S13). The memory 26b stores the maximum likelihood coordinates.

The processor 26a determines whether the candidate vector norm error e₁ is less than a predetermined threshold e₁ₜₕ (first range) (step S14). Here, the memory 26b stores the predetermined threshold e₁ₜₕ. The processor 26a reads the predetermined threshold e₁ₜₕ from the memory 26b and performs the determination in step S14.

Note that as shown in (Equation 10), the candidate vector norm error e₁ takes a value of 0 or more. For this reason, the first range is a range of 0 or more and less than e₁ₜₕ.

Here, if the candidate vector norm error e₁ is not smaller than the predetermined threshold e₁ₜₕ, the processor 26a determines whether the counter j has reached a predetermined value p (step S15). The predetermined value p is a total number of the candidate coordinates in the predetermined spatial range centered on the search center coordinates p0.

For example, in the example shown in FIG. 6, except when the processing shown in FIG. 7 is started and the search center coordinates p0 are selected first, the candidate coordinates are the lattice point coordinates p1 to p26 (as described above, since the search center coordinates p0 selected in the second and subsequent selection are coordinates for which the candidate vector norm error e₁ (or the normalized electromotive voltage error e₂ described later) has already been calculated). Therefore, the processor 26a determines in step S15 whether the processing using all the lattice point coordinates p1 to p26 as the candidate coordinates has been completed depending on whether the counter j has reached the predetermined value p=26.

In step S15, if the counter j has not reached the predetermined value p, the processor 26a increments the counter j (step S16), and proceeds to the processing of step S5 of FIG. 7. In step S5, the processor 26a selects lattice point coordinates that have not yet been selected from among the lattice point coordinates p1 to p26 as new candidate coordinates, and performs the processing described above. Thus, if the candidate vector norm error e₁ is not within the first range, the processor 26a selects the new candidate coordinates.

In addition, in step S15, if the counter j has reached the predetermined value p, the processor 26a determines whether the counter i has reached a predetermined value m1 (step S15A). Here, the predetermined value m1 is more than 0 and less than a predetermined value m described later (see step S28 in FIG. 9). The memory 26b stores the predetermined value m1. The processor 26a reads the predetermined value m1 from the memory 26b, and performs the determination in step S15A.

Here, if the counter i has reached the predetermined value m1, the processor 26a proceeds to the processing of step S18 described later.

In step S15A, if the counter i has not reached the predetermined value m1, the processor 26a increments the counter i and resets the counter j to 0 (step S17), and proceeds to the processing of step S3 of FIG. 7. In step S3, the processor 26a selects three-dimensional coordinates of another point included in the entire range stored in the memory 26b as the search center coordinates p0, and performs the processing described above.

In other words, when the candidate vector norm error e₁ is not within the first range even if every one of the plurality of sets of lattice point coordinates p1 to p26 has been selected as the candidate coordinates, the processor 26a selects new search center coordinates p0.

On the other hand, in step S14, if the candidate vector norm error e₁ is less than the predetermined threshold e₁ₜₕ, the processor 26a enters the value ST2 indicating stage 2 into the parameter ST indicating the current stage (step S18).

Then, the processor 26a updates the search center coordinates p0 to the current maximum likelihood coordinates (step S19), proceeds to the processing of step S4 of FIG. 7, and further performs the processing up to step S6.

Therefore, when the candidate vector norm error e₁ is within the first range in step S14, the processor 26a selects the current maximum likelihood coordinates as the new search center coordinates p0 in step S19, and selects one of the plurality of sets of lattice point coordinates p1 to p26 within the predetermined spatial range centered on the search center coordinates p0 as the candidate coordinates in step S5.

Since the value ST2 is entered into the parameter ST in step S18, in the subsequent step S7, the processor 26a determines that the value of the parameter ST is not ST1, and proceeds to the stage 2 processing of FIG. 9 (step S9).

When entering the stage 2 processing of FIG. 9, the processor 26a (the estimated electromotive voltage arithmetic unit 34) calculates an estimated electromotive voltage BₖGₖ₊₁ (see the second term in the norm of the numerator in the sigma of the right-hand side of (Equation 11)), based on the matrix Bₖ and the candidate vector Gₖ₊₁ (step S21).

Furthermore, the processor 26a (the normalized voltage error arithmetic unit 35) calculates an electromotive voltage error (see the norm of the numerator in the sigma of the right-hand side of (Equation 11)), which is an error between the estimated electromotive voltage BₖGₖ₊₁ and the electromotive voltage vector vₖ (measurement voltage) obtained from the detection signal, normalizes the electromotive voltage error based on the electromotive voltage vector vₖ (measurement voltage), and calculates the normalized electromotive voltage error e₂ expressed by (Equation 11)(step S22).

$${e}_{2} = {\sum }_{k = 1}^{l} \frac{1}{{‖ \vec{{v}_{k}} ‖}^{2}} {‖ \vec{{v}_{k}} - {B}_{k} \vec{{G}_{k + 1}} ‖}^{2} \qquad ( Equation 11 )$$

Note that, in the right-hand side of (Equation 11), the sigma represents the summation performed for k=1 to 1, but the subscript (k+1) of the candidate vector Gₖ₊₁ is treated as “1” when k=1.

Each time a new normalized electromotive voltage error e₂ is calculated, the processor 26a compares the new normalized electromotive voltage error e₂ with a minimum value of the normalized electromotive voltage error e₂ calculated in the past (step S23).

In other words, the processor 26a stores the minimum value of the normalized electromotive voltage error e₂ calculated after starting the processing shown in FIG. 7 in the memory 26b, for example. Then the processor 26a compares the minimum value of the normalized electromotive voltage error e₂ stored in the memory 26b with the newly calculated normalized electromotive voltage error e₂.

Here, if the newly calculated normalized electromotive voltage error e₂ is smaller than the minimum value of the normalized electromotive voltage error e₂ calculated in the past, the processor 26a updates the maximum likelihood coordinates to the current candidate coordinates (step S24). The memory 26b stores the maximum likelihood coordinates.

The processor 26a determines whether the normalized electromotive voltage error e₂ is less than a predetermined threshold e₂ₜₕ (second range) (step S25). Here, the memory 26b stores the predetermined threshold e₂ₜₕ. The processor 26a reads the predetermined threshold e₂th from the memory 26b, and performs the determination in step S25.

Note that, as shown in (Equation 11), the normalized electromotive voltage error e₂ takes a value of 0 or more. For this reason, the second range is a range of 0 or more and less than e₂ₜₕ.

Here, if the normalized electromotive voltage error e₂ is not smaller than the predetermined threshold e₂ₜₕ, the processor 26a determines whether the counter j has reached the predetermined value p (step S26).

In step S26, if the counter j has not reached the predetermined value p, the processor 26a increments the counter j (step S27), and proceeds to the processing of step S5 of FIG. 7. In step S5, the processor 26a selects the lattice point coordinates that have not yet been selected from among the lattice point coordinates p1 to p26 as new candidate coordinates, and performs the processing described above. Thus, if the normalized electromotive voltage error e₂ is not within the second range, the processor 26a selects new candidate coordinates.

In addition, in step S26, if the counter j has reached the predetermined value p, the processor 26a determines whether the counter i has reached the predetermined value m (step S28). Here, the memory 26b stores the predetermined value m. The processor 26a reads the predetermined value m from the memory 26b, and performs the determination in step S28.

In step S28, if the counter i has not reached the predetermined value m, the processor 26a increments the counter i, and resets the counter j to 0 (step S29).

Furthermore, the processor 26a updates the search center coordinates p0 to the current maximum likelihood coordinates (step S30), proceeds to the processing of step S4 of FIG. 7, and further performs the processing of step S5.

Therefore, when the normalized electromotive voltage error e₂ is not within the second range even if every one of the plurality of sets of lattice point coordinates p1 to p26 has been selected as the candidate coordinates, the processor 26a selects the current maximum likelihood coordinates as new search center coordinates p0, and selects one of the plurality of sets of lattice point coordinates p1 to p26 within the predetermined spatial range centered on the search center coordinates p0 as the candidate coordinates.

Note that, as described above, the processor 26a reduces the predetermined spatial range and shortens the lattice point interval when selecting the new search center coordinates p0.

On the other hand, if the normalized electromotive voltage error e₂ is less than the predetermined threshold e₂ₜₕ in step S25 or if the counter i has reached the predetermined value m in step S28, the processor 26a (the estimated coordinate acquisition unit 36) acquires the current maximum likelihood coordinates as the estimated coordinates (step S31).

In other words, if the normalized electromotive voltage error e₂ is within the second range, the processor 26a acquires the current maximum likelihood coordinates as the estimated coordinates of the magnetic field generation element 22.

In addition, in the present embodiment, even if the normalized electromotive voltage error e₂ is not smaller than the predetermined threshold e₂ₜₕ, if the counter i has reached the predetermined value m, the processor 26a acquires the current maximum likelihood coordinates as the estimated coordinates.

Furthermore, the direction vector g indicating the direction of the magnetic field generation element 22 (the direction of the transmission coil C) whose estimated coordinates are acquired is expressed by (Equation 12) as a unit vector by normalizing the vector sum of the candidate vectors Gₖ of 1 sets of the reception coils SCk.

$$\vec{g} = \frac{{\sum }_{k = 1}^{l} \vec{{G}_{k}}}{‖ {\sum }_{k = 1}^{l} \vec{{G}_{k}} ‖} \qquad ( Equation 12 )$$

Here, the candidate vector Gₖ used in (Equation 12) is recalculated using (Equation 4) to (Equation 9) based on the estimated coordinates acquired in step S31 and the x, y, and z coordinates x_{Rk}, y_{Rk}, and z_{Rk} of the reception coils SCk of the set k.

Therefore, the direction vector g (estimated vector) expressed by (Equation 12) is a maximum likelihood vector corresponding to the maximum likelihood coordinates that are the basis for the estimated coordinates.

After performing the processing of step S31, the process is returned to the main processing not shown in the drawings.

According to the first embodiment, in stage 1, the candidate vector Gₖ is brought close to a likely vector, and when the candidate vector norm error e₁ becomes less than the predetermined threshold e₁ₜₕ, the process proceeds to stage 2 to bring the candidate coordinates close to likely coordinates. This can inhibit the candidate vector Gₖ from deviating significantly from the true value and also inhibit the estimated coordinates from falling into a local minimum without approaching the true value.

For example, at a near point, the candidate vector Gₖ tends to deviate from the true value. Here, the near point is a point closest to the magnetic field detection element 23 in a range (entire range) of a space in which positions can be detected by the magnetic field detection element 23. In contrast, in the present embodiment, as shown in (Equation 10), the candidate vector norm error e₁ based on the norm of the vector obtained by dividing the candidate vector Gₖ by the constant ζ (that is, a norm close to a value 1, which is the norm of the unit vector) is used to evaluate likelihood of the candidate vector Gₖ. Therefore, in the present embodiment, the candidate vector norm error e₁ can be accurately determined even at the near point, and the candidate vector Gₖ can be brought close to the true value.

In addition, at the near point, the electromotive voltage becomes large, so even a slight coordinate error may easily cause a large voltage error between the estimated electromotive voltage and the measurement voltage. In contrast, in the present embodiment, as shown in (Equation 11), the electromotive voltage error is normalized based on the measurement voltage, and it is determined whether the normalized electromotive voltage error e₂ is less than the predetermined threshold e₂ₜₕ. Therefore, in the present embodiment, an error rate between the estimated electromotive voltage and the measurement voltage can be accurately determined even at the near point, and the estimated coordinates can be brought close to the true value.

In stage 2 shown in FIG. 9, even if the process returns to the processing of step S4 or step S5 of FIG. 7, the process does not return to the processing of step S3 of FIG. 7. In other words, in stage 2, no other point included in the entire range stored in the memory 26b is selected as the search center coordinates p0. Thus, in stage 2, only the lattice point coordinates p1 to p26, which use the maximum likelihood coordinates obtained in stage 1, as the search center coordinates p0, are searched, so the estimated coordinates can be rapidly acquired with a small number of search points (that is, a small amount of calculation).

In this way, relative positions and directions between the magnetic field generation element 22, which is located in a wide range including the near point of the magnetic field detection element 23, and the magnetic field detection element 23 can be estimated rapidly with a small amount of calculation.

Furthermore, when resetting the search center coordinates p0 and the lattice point coordinates, the estimated coordinates can be acquired with high accuracy since the lattice point interval are made shorter.

In addition, the magnetic field generation element 22 is disposed in the endoscope 3, and the magnetic field detection element 23 is disposed in the antenna 28 outside the endoscope 3. Thus, there is a high degree of freedom in the arrangement of the reception coil groups 23g1 to 23g4 in the magnetic field detection element 23. Therefore, as shown in FIG. 2, the reception coil groups 23g1 to 23g4 can be disposed away from a noise source, for example, thereby improving the accuracy of the detection signal.

### Second Embodiment

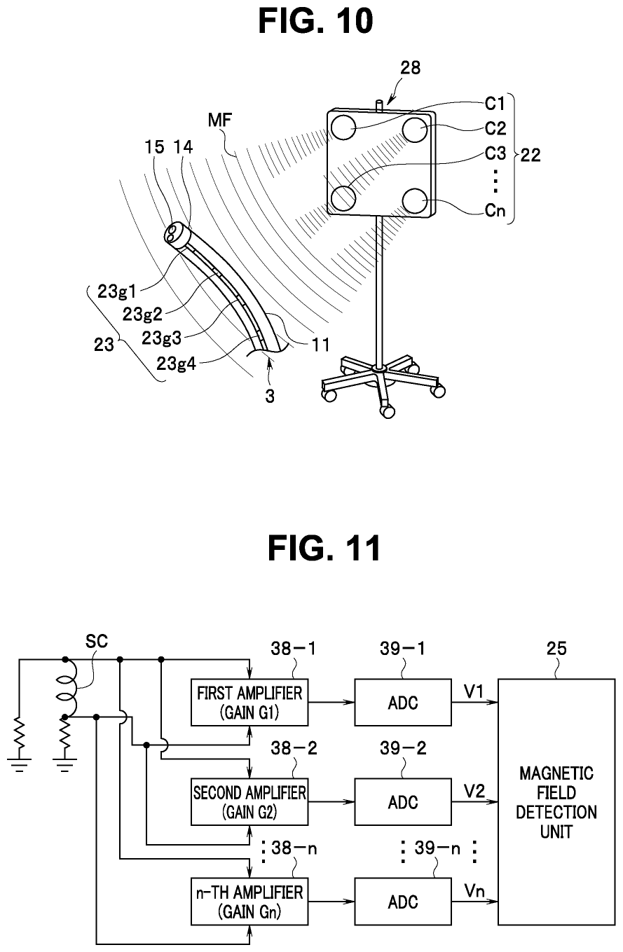

FIG. 10 shows a configuration example in which the magnetic field detection element 23 is disposed in the endoscope 3 and the magnetic field generation element 22 is disposed in the antenna 28 outside the endoscope 3 in a second embodiment of the present disclosure. In the second embodiment, the same parts as those in the first embodiment are attached with the same reference signs, and descriptions thereof are omitted as appropriate. In the second embodiment, differences from the first embodiment are mainly described.

In the first embodiment, the magnetic field generation element 22 is disposed in the endoscope 3, and the magnetic field detection element 23 is disposed in the antenna 28 (the reception antenna in the example of FIG. 2) outside the endoscope 3. In contrast, in the second embodiment, the magnetic field generation element 22 is disposed in the antenna 28 (the transmission antenna in the example of FIG. 10) outside the endoscope 3, and the magnetic field detection element 23 is disposed in the endoscope 3.

In this way, the magnetic field generation element 22 (in a specific example, the transmission coils C1 to Cn) and the magnetic field detection element 23 (in a specific example, the reception coil groups 23g1 to 23g4) may be disposed in a reversed order from that shown in the first embodiment.

According to the second embodiment, the same effects as those in the first embodiment described above are provided.

In addition, in general, a current value of the detection signal transmitted by the magnetic field detection element 23 detecting the magnetic field MF is smaller than a current value of the driving signal transmitted by the driving signal transmission unit 24 to the magnetic field generation element 22. Therefore, according to the second embodiment, an amount of heat generated from the coils in the endoscope 3 can be reduced compared to the first embodiment, and a temperature rise of the endoscope 3 can be suppressed. This reduces, for example, generation of thermal noise of the image pickup device 16 due to the temperature rise. In addition, a burden on a patient due to the temperature rise of the endoscope 3 can also be reduced.

### Third Embodiment

FIG. 11 shows a configuration example in which a signal detected by a reception coil SC of the magnetic field detection element 23 is amplified so as to be able to change an amplification factor, converted into a digital signal, and transmitted to the magnetic field detection unit 25 in a third embodiment of the present disclosure. In the third embodiment, the same parts as those in the first and second embodiments are attached with the same reference signs, and descriptions thereof are omitted as appropriate. In the third embodiment, differences from the first and second embodiments are mainly described.

The position detection system 2 further includes first to n-th amplifiers 38-1 to 38-n that amplify signals generated by the magnetic field detection element 23 detecting the magnetic field MF, and transmit detection signals.

The plurality (n in this case) of amplifiers 38-1, 38-2, . . . , 38-n are connected in parallel to both ends of the reception coil SC. The n amplifiers 38-1, 38-2, . . . , 38-n detect the electromotive voltage at the both ends of the reception coil SC respectively, and amplify the electromotive voltage by respective gains.

If the gain of the first amplifier 38-1 is G1, the gain of the second amplifier 38-2 is G2, . . . , and the gain of the n-th amplifier 38-n is Gn, for example, respective amplifiers are configured such that a relation of G1<G2< . . . <Gn is established.

The first amplifier 38-1 is connected to an ADC 39-1. The ADC 39-1 transmits a digital detection signal including information on a voltage V1 amplified by the first amplifier 38-1 with the gain G1 to the magnetic field detection unit 25.

The second amplifier 38-2 is connected to an ADC 39-2. The ADC 39-2 transmits a digital detection signal including information on a voltage V2 amplified by the second amplifier 38-2 with the gain G2 to the magnetic field detection unit 25.

The other amplifiers are similar, and the n-th amplifier 38-n is connected to an ADC 39-n. The ADC 39-n transmits a digital detection signal including information on a voltage Vn amplified by the n-th amplifier 38-n with the gain Gn to the magnetic field detection unit 25.

The magnetic field detection unit 25 receives the detection signals including the information on the voltages V1 to Vn respectively from the n ADC 39-1 to 39-n. As described above, since the gains have the relation as G1<G2< . . . <Gn, the voltages generally have a relation as V1<V2< . . . <Vn.

The magnetic field detection unit 25 uses threshold voltages V1ₜₕ to Vnₜₕ for each of the voltages V1 to Vn to acquire an electromotive voltage V generated at both ends of the reception coil SC as follows. Here, the threshold voltages V1ₜₕ to Vnₜₕ may be implemented as hardware in the magnetic field detection unit 25, or may be stored in a memory (not shown) in the magnetic field detection unit 25 or in the memory 26b in the control unit 26.

The magnetic field detection unit 25 first compares the voltage V1 with the threshold voltage V1ₜₕ, and when V1>V1ₜₕ, the electromotive voltage Vis calculated as V=V1/G1.

In addition, when V1≤V1ₜₕ, the magnetic field detection unit 25 compares the voltage V2 with the threshold voltage V2ₜₕ, and when V2>V2ₜₕ, the electromotive voltage V is calculated as V=V2/G2.

When V2≤V2ₜₕ, the magnetic field detection unit 25 performs the same processing sequentially for the voltage V3 and thereafter, and when V(n−1)≤V(n−1)ₜₕ, the magnetic field detection unit 25 compares the voltage Vn with the threshold voltage Vnₜₕ, and the electromotive voltage V is calculated as V=Vn/Gn when Vn>Vnₜₕ.

Note that when Vn≤Vnₜₕ, the magnetic field detection unit 25 may transmit an error signal to the control unit 26. In this case, the control unit 26 assumes that the magnetic field MF is not detected by the magnetic field detection unit 25 and stops performing the processing shown in FIG. 7 until the detection signal is next received from the magnetic field detection unit 25.

Alternatively, even when Vn≤Vnₜₕ, the magnetic field detection unit 25 may calculate the electromotive voltage V as V=Vn/Gn.

As described above, the reason for checking the voltage in the order of V1, V2, . . . , Vn is that if the amplification factor is too large for the magnitude of the signal, the voltage value exceeds an upper limit value of a detectable range of the ADC, and the voltage cannot be detected correctly.

In this way, the first to n-th amplifiers 38-1 to 38-n function as amplification units in which the amplification factor can be changed depending on the voltage of the signal.

Note that FIG. 11 shows the example in which the plurality of amplifiers 38 with different gains and the plurality of ADCs 39 connected to the plurality of amplifiers 38 respectively, but it is not limited to this configuration. For example, one amplifier 38 with a variable gain and one ADC 39 connected to the amplifier 38 are provided, and the gain of the amplifier 38 may be changed depending on the voltage of the signal transmitted from the reception coil SC.

In addition, the current value of the driving signal transmitted by the driving signal transmission unit 24 to the magnetic field generation element 22 (and thus the strength of the magnetic field MF generated by the magnetic field generation element 22) may be changed so that the voltage of the signal transmitted from the reception coil SC falls within a certain voltage range.

According to the third embodiment, the same effects as those in the first and second embodiments as described above are provided. Furthermore, the third embodiment provides the following effects.

The ADC 39 shown in FIG. 4 has a voltage detectable range (dynamic range) and resolution limitations. For this reason, even if an algorithm is improved, it may not be possible to detect the position of the magnetic field generation element 22 located in a wide range from the near point to a far point. Here, the far point is a point furthest from the magnetic field detection element 23 in the range (entire range) of the space in which the position can be detected by the magnetic field detection element 23.

In contrast, according to the third embodiment, since the amplification factor of the amplifiers can be changed depending on the voltage of the signal generated by the magnetic field detection element 23 detecting the magnetic field MF, the detectable voltage range is expanded. Alternatively, since the current value of the driving signal can be changed so that the voltage of the signal generated by detecting the magnetic field MF falls within a certain voltage range, the magnetic field MF can always be detected. This makes it possible to detect the position of the magnetic field generation element 22 located in the wide range from the near point to the far point, with high accuracy.

### Fourth Embodiment

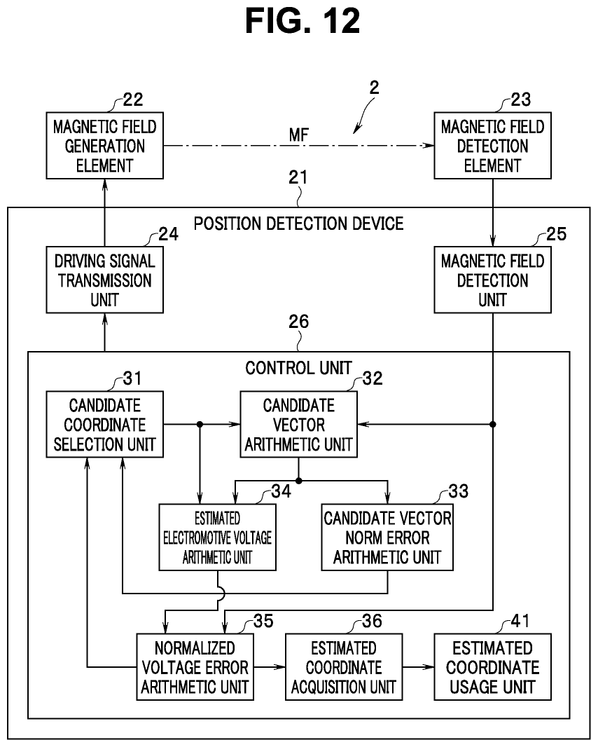

FIG. 12 to FIG. 14 show a fourth embodiment of the present disclosure. In the fourth embodiment, the same parts as those in the first to third embodiments are attached with the same reference signs, and descriptions thereof are omitted as appropriate. In the fourth embodiment, differences from the first to third embodiments are mainly described.

FIG. 12 shows a configuration of a position detection system 2 in the fourth embodiment.

A position detection device 21 shown in FIG. 12 further includes, in addition to the configuration shown in FIG. 5, an estimated coordinate usage unit 41 connected to the estimated coordinate acquisition unit 36. The estimated coordinate usage unit 41 is, for example, provided as a function unit in the control unit 26. However, the estimated coordinate usage unit 41 may be provided outside the control unit 26.

The estimated coordinate usage unit 41 receives the estimated coordinates (and, if necessary, estimated vectors, the same applies hereinafter) transmitted from the estimated coordinate acquisition unit 36. The estimated coordinate usage unit 41 generates a shape detection image indicating a shape of the insertion portion 11, for example, based on the received estimated coordinates of the transmission coils C1 to Cn. In addition, the estimated coordinate usage unit 41 may generate insertion assistance information based on the received estimated coordinates.

The shape detection image, the insertion assistance information, and the like generated by the estimated coordinate usage unit 41 are transmitted to the video processor 5 connected to the position detection device 21, and displayed on the monitor 6 together with the endoscopic image.

Note that, although not explicitly described, the position detection system 2 of the above described first to third embodiments may include the estimated coordinate usage unit 41.

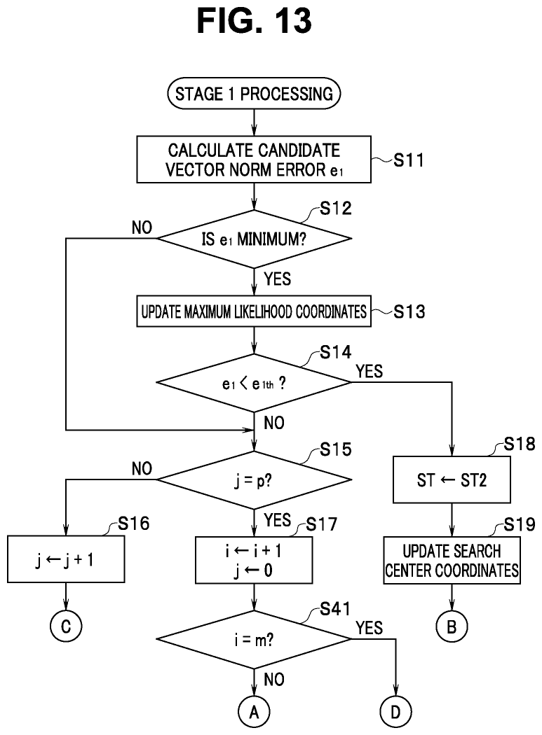

FIG. 13 is a flowchart showing stage 1 processing, which is a part of transmission coil coordinate estimation processing in the fourth embodiment.

The stage 1 processing shown in FIG. 13 is basically the same as the stage 1 processing shown in FIG. 8, but after performing the processing of step S17, the processor 26a further determines whether the counter i has reached the predetermined value m (step S41).

Here, if the counter i has not reached the predetermined value m, the processor 26a proceeds to the processing of step S3 of FIG. 7.

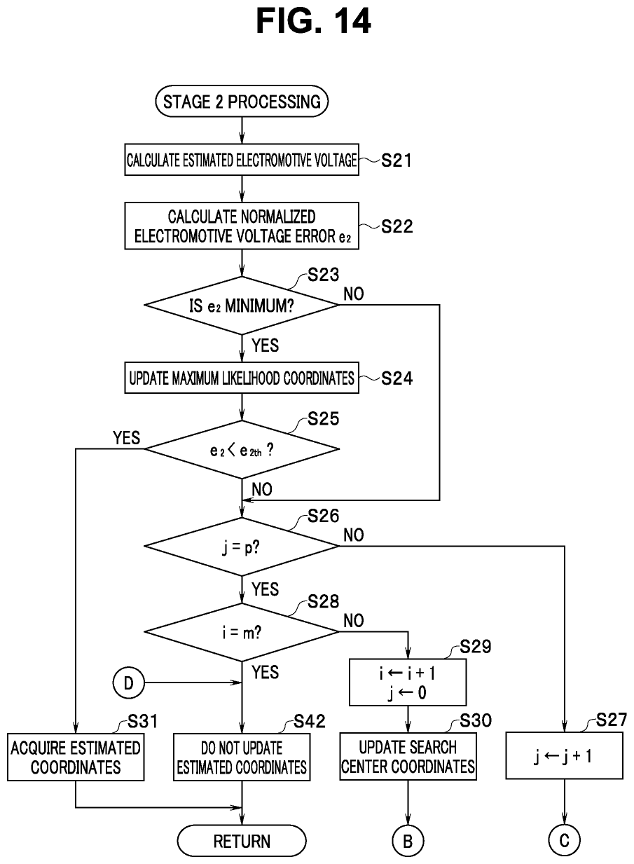

On the other hand, if the counter i has reached the predetermined value m in step S41, the processor 26a proceeds to the processing of step S42 of FIG. 14 described later.

FIG. 14 is a flowchart showing stage 2 processing, which is a part of the transmission coil coordinate estimation processing in the fourth embodiment.

The stage 2 processing shown in FIG. 14 is basically the same as the stage 2 processing shown in FIG. 9, but if the counter i has reached the predetermined value m in step S28, the processor 26a does not update the estimated coordinates (step S42). In addition, as described above, even if the counter i has reached the predetermined value m in step S41 of the stage 1 processing, the processor 26a does not update the estimated coordinates in step S42.

In this way, the processor 26a counts the number of times the search center coordinates p0 are selected using the counter i, and if the candidate vector norm error e₁ is not within the first range or the normalized electromotive voltage error e₂ is not within the second range even if the count value has reached the predetermined value m, the processor 26a does not acquire the estimated coordinates of the magnetic field generation element 22.

Therefore, the estimated coordinates are acquired (updated) by the processing of step S31 when, after the transmission coil coordinate estimation processing of FIG. 7 is called in the main processing, the candidate vector norm error e₁ is less than the predetermined threshold e₁ₜₕ in the step S14 of the stage 1 processing of FIG. 13, and the normalized electromotive voltage error e₂ is less than the predetermined threshold e₂th in step S25 of the stage 2 processing of FIG. 14.

When the endoscope 3 is being moved, there is a possibility that the conditions e₁<e₁ₜₕ and e₂<e₂th cannot be satisfied instantaneously, making it difficult to estimate the position and the direction. In addition, similarly, when electromagnetic noise occurs, or when there are individual variations in the coils of the transmission and reception system that transmits and receives the magnetic field MF, in the constant, or the like, it may also be difficult to estimate the position and the direction.

Therefore, in the present embodiment, if the conditions e₁<e₁ₜₕ and e₂<e₂th cannot be satisfied even after searching the estimated coordinates a predetermined number of times (the number of times until the counter i reaches the predetermined value m), the estimated coordinates are not updated. In this case, the estimated coordinates acquired when the transmission coil coordinate estimation processing of FIG. 7 is last called in the main processing are continued to be used.

According to the fourth embodiment, the same effects as those in the first to third embodiments described above are provided.

In addition, according to the fourth embodiment, since the estimated coordinates are not updated when the estimated coordinates that are estimated to be correct cannot be acquired, the estimated coordinate usage unit 41 can be inhibited from generating an inaccurate shape detection image or inaccurate insertion assistance information.

### Fifth Embodiment

A fifth embodiment of the present disclosure will be described with reference to FIG. 1 and the like. In the fifth embodiment, the same parts as those in the first to fourth embodiments are attached with the same reference signs, and descriptions thereof are omitted as appropriate. In the fifth embodiment, differences from the first to fourth embodiments are mainly described.

The value of the constant ζ used to calculate the candidate vector norm error e₁ is determined depending on the gain of the entire transmission and reception system including the transmission system that transmits the magnetic field MF and the reception system that receives the magnetic field MF, as described above. Therefore, the value of the constant ζ may vary due to the individual differences on the endoscope 3 side where the magnetic field generation element 22 (or the magnetic field detection element 23) is provided, and the value of the constant ζ may also vary due to the individual differences on the position detection system 2 side where the magnetic field detection element 23 (or the magnetic field generation element 22) is provided.

Therefore, a first correction value (individual adjustment value) for the constant ζ is stored in the memory 17 provided in the endoscope 3, to correct an influence on the candidate vector norm error e₁ due to the individual variations on the endoscope 3 side.

Furthermore, a second correction value (individual adjustment value) for the constant ζ is stored in the memory 26b provided in the position detection device 21, for example, to correct an influence on the candidate vector norm error e₁ due to the individual variations on the position detection system 2 side.

The processor 26a of the position detection device 21 corrects the constant ζ based on the first correction value read from the memory 17 of the endoscope 3 and the second correction value read from the memory 26b of the position detection device 21, and calculates the candidate vector norm error e₁ using the corrected constant ζ.

According to the fifth embodiment, the same effects as those in the first to fourth embodiments described above are provided.

According to the fifth embodiment, the individual adjustment values of the constant ζ used in the process of calculating the candidate vector norm error e₁ are stored in the memory on the side that transmits the magnetic field MF and the memory on the side that receives the magnetic field MF. Therefore, even if the individual variations are large, the estimated position can be acquired stably and with high accuracy.

Furthermore, even when different types of endoscopes 3 are combined with the position detection system 2 (or when the endoscope 3 is combined with different types of position detection systems 2), the estimated position can be acquired stably and with high accuracy.

Note that in the above description, the case where the present disclosure relates to the control device or the endoscope system has been mainly described, but the present disclosure is not limited thereto. For example, the present disclosure may relate to a position detection method that performs the same processing as the control device. In addition, the present disclosure may relate to a computer program for causing a computer to perform the same processing as the control device. Furthermore, the present disclosure may be a non-transitory computer-readable storage medium that stores the computer program, or the like.

Here, examples of the storage medium storing a computer program product may include, but are not limited to, portable storage media such as a flexible disk, a compact disc read only memory (CD-ROM), a digital versatile disc (DVD), and a universal serial bus (USB) memory, and storage media such as a hard disk drive (HDD) and a solid state drive (SSD). The storage medium may not necessarily store the entirety of the computer program but may store a part of the computer program. In addition, at least a part of the computer program may also be distributed or provided via a communication network. When a user installs the computer program on the computer, for instance, from the storage medium or after downloading the computer program via the communication network, the user may cause the computer to read the computer program and execute at least one of the operations, thereby achieving the operations of the control device as described above.

Furthermore, the present disclosure is not limited to the above-described embodiments as they are. The present disclosure can be embodied by modifying the constituent elements within the scope not departing from the gist of the disclosure at the stage of implementation. Furthermore, various forms of the disclosure can be formed by appropriately combining a plurality of constituent elements disclosed in the above embodiments. For example, some constituent elements may be deleted from all constituent elements disclosed in the embodiments. Furthermore, the constituent elements in the different embodiments may be combined as appropriate. In this way, it is obvious that various modifications and applications are possible within the scope not departing from the gist of the disclosure.

### (General Interpretation Notes)

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, or other storage media) that stores data, software, or instructions contributing to the processing operations. Accordingly, the processing circuitry may be implemented as a specialized hardware circuit having fixed logic, a programmable circuit executing instructions stored in an internal or external memory, or any combination thereof. Furthermore, like the “processor” described above, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: A control device comprising a processor, wherein
the processor is configured to:
   select candidate coordinates of a magnetic field generation element;
   calculate a candidate vector based on coordinates of a magnetic field detection element, the candidate coordinates, and a detection signal transmitted by the magnetic field detection element detecting a magnetic field generated by the magnetic field generation element;
   calculate a vector norm error based on the candidate vector;
   update maximum likelihood coordinates to the candidate coordinates currently selected, when the calculated vector norm error is smaller than a minimum value of the vector norm error calculated in a past;
   determine whether the vector norm error is within a first range; and
   newly select the candidate coordinates when the vector norm error is not within the first range.

Clause 2: The control device according to clause 1, wherein the processor is configured to select one of a plurality of sets of lattice point coordinates within a predetermined spatial range centered on search center coordinates, as the candidate coordinates.

Clause 3: The control device according to clause 2, wherein the processor is configured to select the new search center coordinates when the vector norm error is not within the first range even if every one of the plurality of sets of lattice point coordinates has been selected as the candidate coordinates.

Clause 4: The control device according to clause 2, wherein
the processor is configured to:
   when the vector norm error is within the first range,
      select the current maximum likelihood coordinates as the new search center coordinates; and
      select one of the plurality of sets of lattice point coordinates within the predetermined spatial range centered on the search center coordinates, as the candidate coordinates.

Clause 5: The control device according to clause 4, wherein the processor is configured to reduce the predetermined spatial range and shorten a lattice point interval when the new search center coordinates are selected.

Clause 6: The control device according to clause 4, wherein
the processor is configured to:
when the vector norm error is within the first range,
   calculate a normalized electromotive voltage error based on the candidate vector; and
   update the maximum likelihood coordinates to the candidate coordinates currently selected, when the calculated normalized electromotive voltage error is smaller than a minimum value of the normalized electromotive voltage error calculated in the past;
determine whether the normalized electromotive voltage error is within a second range; and
newly select the candidate coordinates when the normalized electromotive voltage error is not within the second range.

Clause 7: The control device according to clause 6, wherein
the processor is configured to:
   calculate an estimated electromotive voltage based on the candidate vector;
   calculate an electromotive voltage error, which is an error between the estimated electromotive voltage and a measurement voltage obtained from the detection signal; and
   calculate the normalized electromotive voltage error by normalizing the electromotive voltage error based on the measurement voltage.

Clause 8: The control device according to clause 6, wherein
the processor is configured to:
   when the normalized electromotive voltage error is not within the second range even if every one of the plurality of sets of lattice point coordinates has been selected as the candidate coordinates, perform:
      selecting the current maximum likelihood coordinates as the new search center coordinates; and
      selecting one of the plurality of sets of lattice point coordinates within the predetermined spatial range centered on the search center coordinates as the candidate coordinates.

Clause 9: The control device according to clause 8, wherein the processor is configured to reduce the predetermined spatial range and shorten a lattice point interval when the new search center coordinates are selected.

Clause 10: The control device according to clause 6, wherein the processor is configured to acquire the current maximum likelihood coordinates as estimated coordinates of the magnetic field generation element when the normalized electromotive voltage error is within the second range.

Clause 11: The control device according to clause 8, wherein the processor is configured not to acquire estimated coordinates of the magnetic field generation element when a number of times the search center coordinates are selected is counted, and the vector norm error is not within the first range or the normalized electromotive voltage error is not within the second range even if a count value has reached a predetermined value.

Clause 12: An endoscope system comprising:
an endoscope;
a magnetic field generation element configured to generate a magnetic field;
a magnetic field detection element configured to detect the magnetic field and transmit a detection signal; and
a processor configured to process the detection signal, wherein
the processor is configured to:
   select candidate coordinates of the magnetic field generation element;
   calculate a candidate vector based on coordinates of the magnetic field detection element, the candidate coordinates, and the detection signal;
   calculate a vector norm error based on the candidate vector;
   update maximum likelihood coordinates to the candidate coordinates currently selected, when the calculated vector norm error is smaller than a minimum value of the vector norm error calculated in a past;
   determine whether the vector norm error is within a first range; and
   newly select the candidate coordinates when the vector norm error is not within the first range.

Clause 13: The endoscope system according to clause 12, further comprising:
an amplifier configured to amplify a signal generated by the magnetic field detection element detecting the magnetic field, and transmit the detection signal; wherein
the amplifier can change an amplification factor depending on a voltage of the signal.

Clause 14: The endoscope system according to clause 12, wherein:
the magnetic field generation element is disposed in the endoscope; and
the magnetic field detection element is disposed outside the endoscope.

Clause 15: The endoscope system according to clause 12, wherein:
the magnetic field generation element is disposed outside the endoscope; and
the magnetic field detection element is disposed in the endoscope.

Clause 16: The endoscope system according to clause 12, wherein the endoscope includes a memory configured to store a first correction value to correct an influence on the vector norm error due to individual variations of the endoscope.

Clause 17: The endoscope system according to clause 12, wherein:
the magnetic field generation element, the magnetic field detection element, and the processor are provided in a position detection system; and
the position detection system includes a memory configured to store a second correction value to correct an influence on the vector norm error due to individual variations of the position detection system.

Clause 18: A position detection method comprising:
generating a magnetic field by a magnetic field generation element;
detecting the magnetic field and transmitting a detection signal by a magnetic field detection element;
selecting candidate coordinates of the magnetic field generation element;
calculating a candidate vector based on coordinates of the magnetic field detection element, the candidate coordinates, and the detection signal;
calculating a vector norm error based on the candidate vector;
updating maximum likelihood coordinates to the candidate coordinates currently selected, when the calculated vector norm error is smaller than a minimum value of the vector norm error calculated in a past;
determining whether the vector norm error is within a first range; and
newly selecting the candidate coordinates when the vector norm error is not within the first range.

Clause 19: The position detection method according to clause 18, further comprising selecting one of a plurality of sets of lattice point coordinates within a predetermined spatial range centered on search center coordinates, as the candidate coordinates.

Clause 20: The position detection method according to clause 19, further comprising selecting the new search center coordinates when the vector norm error is not within the first range even if every one of the plurality of sets of lattice point coordinates has been selected as the candidate coordinates.

What is claimed is:

1. A control device comprising:
a connection interface; and
processing circuitry configured to:
select candidate coordinates of a target element, wherein the target element is one of a magnetic field generation element or a magnetic field detection element;
calculate a candidate vector based on coordinates of a reference element, the candidate coordinates, and a detection signal received via the connection interface from the magnetic field detection element in response to detection of a magnetic field generated by the magnetic field generation element, wherein the reference element is the other of the magnetic field generation element or the magnetic field detection element;
calculate a vector norm error based on the candidate vector;
update maximum likelihood coordinates to the candidate coordinates when the vector norm error is smaller than a minimum value of one or more previously-calculated vector norm errors;
determine whether the vector norm error is within a first range; and
select new candidate coordinates when the vector norm error is not within the first range.

2. The control device according to claim 1, wherein
the processing circuitry is further configured to select, as the candidate coordinates, one of a plurality of sets of lattice point coordinates within a predetermined spatial range centered on search center coordinates.

3. The control device according to claim 2, wherein
the processing circuitry is further configured to select new search center coordinates when the vector norm error is not within the first range even if every one of the plurality of sets of lattice point coordinates has been selected as the candidate coordinates.

4. The control device according to claim 2, wherein
the processing circuitry is further configured to, when the vector norm error is within the first range, perform:
selecting the maximum likelihood coordinates as new search center coordinates; and
selecting, as the candidate coordinates, one of a plurality of sets of lattice point coordinates within an updated spatial range centered on the new search center coordinates.

5. The control device according to claim 4, wherein
the processing circuitry is further configured to, in response to selecting the new search center coordinates, set the updated spatial range by reducing the predetermined spatial range, thereby shortening a lattice point interval.

6. The control device according to claim 4, wherein
the processing circuitry is further configured to, when the vector norm error is within the first range, perform:
calculating a normalized electromotive voltage error based on the candidate vector;
updating the maximum likelihood coordinates to the candidate coordinates when the normalized electromotive voltage error is smaller than a minimum value of one or more previously-calculated normalized electromotive voltage errors;
determining whether the normalized electromotive voltage error is within a second range; and
selecting new candidate coordinates when the normalized electromotive voltage error is not within the second range.

7. The control device according to claim 6, wherein
the processing circuitry is further configured to:
calculate an estimated electromotive voltage based on the candidate vector;
calculate an electromotive voltage error that is an error between the estimated electromotive voltage and a measurement voltage obtained from the detection signal; and
calculate the normalized electromotive voltage error by normalizing the electromotive voltage error based on the measurement voltage.

8. The control device according to claim 6, wherein
the processing circuitry is further configured to, when the normalized electromotive voltage error is not within the second range even if every one of the plurality of sets of lattice point coordinates has been selected as the candidate coordinates, perform:
selecting the current maximum likelihood coordinates as new search center coordinates; and
selecting, as the candidate coordinates, one of a plurality of sets of lattice point coordinates within an updated spatial range centered on the new search center coordinates.

9. The control device according to claim 8, wherein
the processing circuitry is further configured to, in response to selecting the new search center coordinates, set the updated spatial range by reducing the predetermined spatial range, thereby shortening a lattice point interval.

10. The control device according to claim 6, wherein
the processing circuitry is further configured to, when the normalized electromotive voltage error is within the second range, acquire the maximum likelihood coordinates as estimated coordinates of the target element.

11. The control device according to claim 8, wherein
the processing circuitry is further configured to:
count a number of times the search center coordinates are selected; and
not acquire estimated coordinates of the target element when the vector norm error is not within the first range or the normalized electromotive voltage error is not within the second range, even if the counted number of times has reached a predetermined value.

12. The control device according to claim 1, wherein
the processing circuitry comprises:
a processor; and
a non-transitory computer-readable storage medium storing computer-readable instructions configured to, when executed by the processor, cause the processor to:
select the candidate coordinates of the target element;
calculate the candidate vector based on the coordinates of the reference element, the candidate coordinates, and the detection signal received via the connection interface from the magnetic field detection element in response to detection of the magnetic field generated by the magnetic field generation element;
calculate the vector norm error based on the candidate vector;
update the maximum likelihood coordinates to the candidate coordinates when the vector norm error is smaller than the minimum value of the one or more previously-calculated vector norm errors;
determine whether the vector norm error is within the first range; and
select the new candidate coordinates when the vector norm error is not within the first range.

13. An endoscope system comprising:
an endoscope;
a magnetic field generation element configured to generate a magnetic field;
a magnetic field detection element configured to detect the magnetic field and output a detection signal; and
the control device according to claim 1.

14. The endoscope system according to claim 13, further comprising amplification circuitry configured to amplify the detection signal output from the magnetic field detection element and to set an amplification factor depending on a voltage of the detection signal.

15. The endoscope system according to claim 13, wherein
the magnetic field generation element is disposed inside the endoscope, and
the magnetic field detection element is disposed outside the endoscope.

16. The endoscope system according to claim 13, wherein
the magnetic field generation element is disposed outside the endoscope, and
the magnetic field detection element is disposed inside the endoscope.

17. The endoscope system according to claim 13, further comprising a memory configured to store a correction value for correcting an influence of individual variations of the endoscope on the vector norm error.

18. The endoscope system according to claim 13, further comprising a memory configured to store a correction value for correcting an influence of individual variations of a position detection system on the vector norm error, the position detection system comprising the magnetic field generation element, the magnetic field detection element, and the processing circuitry.

19. A position detection method comprising:
generating a magnetic field by a magnetic field generation element;
outputting a detection signal from a magnetic field detection element in response to detection of the magnetic field;
selecting candidate coordinates of a target element, wherein the target element is one of the magnetic field generation element or the magnetic field detection element;
calculating a candidate vector based on coordinates of a reference element, the candidate coordinates, and the detection signal, wherein the reference element is the other of the magnetic field generation element or the magnetic field detection element;
calculating a vector norm error based on the candidate vector;
updating maximum likelihood coordinates to the candidate coordinates when the vector norm error is smaller than a minimum value of one or more previously-calculated vector norm errors;
determining whether the vector norm error is within a first range; and
selecting new candidate coordinates when the vector norm error is not within the first range.

20. The position detection method according to claim 19, wherein
the selecting of the candidate coordinates includes selecting, as the candidate coordinates, one of a plurality of sets of lattice point coordinates within a predetermined spatial range centered on search center coordinates.
